(19) United States
(12) Patent Application Publication
(10) Pub. No.: US 2008/0044480 A1
(43) Pub. Date: Feb. 21, 2008

(54) Technical process and plant for the production of coacervate capsules

(75) Inventor: Rainer Pommersheim, KUPFERBERGTERRASSE 21, 55116 Mainz (DE)

Correspondence Address: BODNER & O'ROURKE, LLP, 425 BROADHOLLOW ROAD, SUITE 108, MELVILLE, NY, 11747, US

(73) Assignee: CAVIS MICROCAPS GMBH, MAINZ GERMANY (DE)

(21) Appl. No.: 10/593,226

(22) PCT Filed: Mar. 4, 2005
(86) PCT No.: PCT/EP05/02324

§ 371 Date: Aug. 3, 2007

(30) Foreign Application Priority Data
Mar. 19, 2004 (DE) 10-2004-013-977.6

(51) Int. Cl. A61K 9/50; B01J 13/04

(52) U.S. Cl. 424/490; 264/4.1

(57) ABSTRACT

The invention relates to a process and the corresponding plant for the production of micro-capsules on an industrial scale for the application in foodstuffs technology, chemical and/or pharmaceutical industry, and in medicine. Said capsules are produced by the so-called coacervate method. They may contain non-living ingredients, e.g. solids, liquids, etc. and also living cells or micro organisms such as bacteria, for example.

The invention relates to a process and the appropriate plant for the production of micro-capsules on an industrial scale for the application in foodstuffs technology, biotechnology, chemical and/or pharmaceutical industry, and in medicine. Said capsules are produced by a so-called coacervate method. They may contain both non-living ingredients, such as solids, liquids etc., and also living cells or micro organisms, such as e. g. bacteria.

In the technological practice but also in medicine, it is often necessary to immobilise solids or liquids, but also living ingredients such as e. g. bacteria. This may be done for purely economic reasons because expensive active substances may be recovered in this manner, but it may also be necessary for process technical reasons, because sensitive ingredients may thereby be protected against the surrounding medium.

In foodstuffs technology, for example, there are cases where substances which are sensitive to oxygen and/or moisture are added to some products. If these ingredients are not protected against the normally oxygen-rich and/or moist surrounding medium they will be oxidised which considerably reduces the shelf life of the products. Such ingredients may, e. g. be artificial aromas or also solids such as iron, fillers, living bacteria etc. In order to ensure that these ingredients meet the specifications until the end of the shelf life of the foodstuffs, the period is either selected relatively short or the doses of the substances are set correspondingly higher.

In other cases it is e. g. necessary to use substances in media with which they react, which in turn would lead to their destruction. It is therefore desirable to bring such substances in contact with the surrounding media with a time offset, i. e. immediately prior to application, in order to ensure their maximum efficiency. Such ingredients may, for example, be active substances contained in cosmetics, which develop their effects only upon skin contact, but also aromas which are released only when chewing the foodstuffs.

In order to be able to encapsulate cells, enzymes, or other substances they are generally added to a liquid, in most cases a water-soluble, basic substance which is then dropped by suitable devices. The formed drops are cured and seal the substance which is solved or suspended therein or the cells. This may either be done by cross-linking in a precipitating bath or by changing physical parameters, such as e. g. the temperature. The pellets formed in this manner may subsequently be coated, which offers a number of further advantages in terms of shelf life or permeability and stability of the pellets. Due to the fact that the first step, i. e. dropping of the basic substance is carried out by means of nozzle systems, it is very difficult to obtain very small pellets in this manner.

An alternative is provided by methods which do not rely on nozzles. The so-called coacervate method is one of them. In accordance with this method, very small particles without additional membrane envelope are obtained.

The coacervate method is based on the following consideration: With the combination of at least two suitable biopolymers in solution, a phase separation may occur by an appropriate modification of the reaction conditions. Therein, a polymer-rich phase, the gel, is separated from a polymer-poor phase, the sol. This process is called the coacervate formation. If the polymers are polyelectrolytes of opposite charge, the process is termed a complex coacervation.

A suitable polymer pair is e. g. gelatine/gum arabic. It is employed for the micro encapsulation of fragrances, dyes, or oils. The material to be encapsulated is emulsified as hydrophobic phase in the polymer solution. The developing coacervate precipitates on the oil droplets and forms the desired capsules with the oil being the core. With a favourable selection of the parameters, capsules with a diameter down to a few micrometers can be obtained.

Presently, a number of methods are known which utilise this process. There are also several commercially available products which are manufactured in accordance with such a method. The most popular in this respect is the carbon paper. Here, small ink-filled pellets are generated in a coacervate process, which are then applied to a foil. With pressure applied to the foil, a number of these pellets burst and release the ink.

Numerous examples of microcapsules are described in the literature, which are produced in a coacervate process. The unexamined application DE 196 44 343 A1, for example, describes a tasteless microcapsule with a diameter of a fewμm, which is produced in an emulsion process and which may be used as a food or animal feed additive as well as a transport system for drugs. Here, oils or substances which are soluble in said oil are emulsified in a basic substance, e. g. alginate, and in a further emulsifying process capsules of 0.5 to 20 μm are formed therefrom which may then be employed in the foodstuffs or pharmaceutical industry. Because of the lack of an additional coating, these pellets, however, cannot be employed in citrate-containing media because citrate would destroy the alginate envelope of these capsules. No technical process is described which would enable a large-scale production of the pellets.

U.S. Pat. No. 5,035,844 describes a coacervate process for the manufacture of pressure sensitive carbon papers. Here, a combination of gelatine, carboxymethyl cellulose and a second anionic polymer is used, such as e. g. a polymethyl methylether/maleic anhydride copolymer. The capsules are not suited for the immobilisation of sensitive materials, not to speak of living ingredients. Here again, no technical process is described for their manufacture.

U.S. Pat. No. 4,376,113 shows a coacervate method for the manufacture of a light and temperature stable capsule. Here, gelatine, gum arabic, ethylhydroxyethyl cellulose are employed. The capsules are cured with glutardialdehyde and may be dried. These capsules as well are hardly suited for the immobilisation of sensitive or living ingredients. Moreover, the technical process for their manufacture is not the subject of the patent.

The majority of these methods utilises toxic substances, or is completely unsuited for biotechnological products, not to speak of foodstuffs, according to the conditions. Moreover, the capsules are not subjected to an additional coating treatment, and technical processes whose subject is coated coacervate capsules are not known.

In view of this situation, the invention is based on the object to describe a method and the associated plant which, for the first time, enable the manufacture of large quantities of coacervate capsules, i. e. the large-scale manufacture, which capsules may be provided with an additional multi-layer membrane envelope, if required, in the same process.

The inventive production process is divided in two sections, the forming and the coating section.

During forming, the material to be encapsulated is suspended in a liquid substance which is not mixable with water, e. g. in a fat or an oil. Subsequently, particles are made in an emulsion process under the addition of substances such as water, gelatine, alginate, glycerine, and a precipitation reagent e. g. calcium chloride, which contain the material to be encapsulated in their interior.

Coating of the gel particles which have been made in this manner is effected by immersion into the respective coating solutions. These are thin aqueous solutions of polymers with anionic or cationic groups such as e. g. chitosan, polyvinyl pyrrolydon, polyethylenimine, carboxymethyl cellulose, alginate, polyacrilic acid, etc., which form so-called polyelectrolyte complex layers on the capsule surface. By repeated immersion of the particles into these solutions several layers of the capsule envelope are formed as described in P 43 12 970.6. In order to prevent the pellets from sticking together during coating and to ensure an optimum membrane formation, the pellets have to be maintained in suspension. According to the invention, this may be done with special agitators, so-called visco-jet agitators, but it is also possible to introduce the coating reagents tangentially at a high velocity into the reactor so that similar to a hydrocyclone a movement of the liquid is achieved which swirls the capsules. Additionally, washing with a suitable detergent may be carried out in between times. The required coating or washing solutions, respectively, are held in reservoirs and may be provided either ready for use or as concentrates.

The production process is carried out at temperatures ranging from 10° to 50° C. and under atmospheric pressure. For this reason, some of the reservoirs which are used in the process must be provided with a temperature equalising capability.

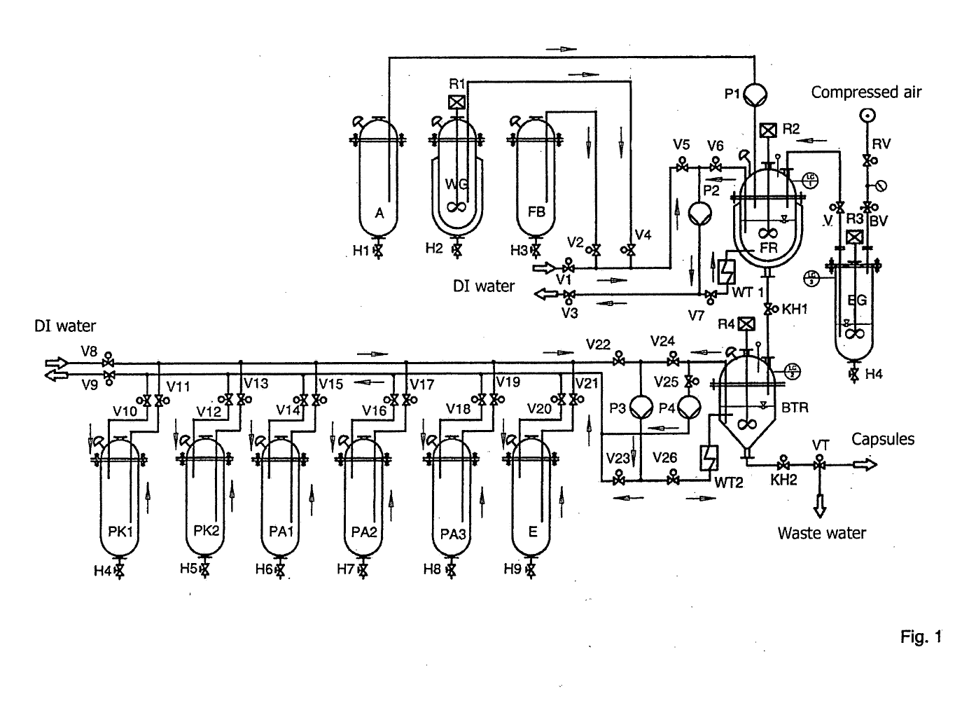

FIG. 1 shows a variant of a method as well as the associated plant for the large-scale production of coacervate capsules which may subsequently be provided with a multi-layer envelope in the same process.

Further variants are, of course, also conceivable such as e. g. a plant which employs one reactor instead of the illustrated two reactors.

The configuration with two reactors has the distinguishing feature of a higher productivity because coating of the pellets can be carried out while dropping of the liquid, i. e. forming is continuing.

Variants with one reactor consequently have a lower productivity, but are simpler and can be easier realised in terms of equipment expenditure.

The technical process shown in FIG. 1 is divided into two sections: The manufacture of uncoated particles, and the coating of said pallets. Depending on the requirements, both the uncoated and the coated particles may be used and further processed. The process is organised as follows:

In a first step, material to be encapsulated is solved, suspended, or emulsified in a liquid which cannot be mixed with water (e. g. oil or fat) in the vessel EG. If, for example, a fat with a higher melting point is used which has first to be heated in order to become liquid, EG has to be provided with a heater or a heating jacket. EG is equipped with an agitator which has to be designed in such a manner that it is able to generate both solutions or suspensions and emulsions.

A solution consisting of water, gelatine, and e. g. glycerine is prepared at a temperature of approx. 50° to 60° C. in the vessel WG which is also equipped with a heater and an agitator. This solution in then delivered into the reactor FR by means of the pump P2 via the valves V4, V5, and V7. The solution inside the reactor FR is maintained at a temperature of approx. 50° to 60° C. by the heat exchanger WT1 and the reactor's jacket or another temperature equalising device.

Subsequently, EG is subjected to compressed air via the valves RV and BV. By opening of the valve V the solution, suspension, or emulsion enters the reactor FR as phase which cannot be mixed with water. With the aid of the agitator R2 a new emulsion is prepared in the reactor FR, while a temperature of approx. 50° to 60° C. is maintained. An Na alginate solution from the reservoir A is then slowly metered into this new emulsion via the metering pump P1.

In a further step the mixture in the reactor FR is cooled down to approx. 10° to 20° C., and a precipitation reagent e. g. an aqueous calcium chloride solution from the reservoir FB is added to the mixture in FR via the valves V2, V5, V7 by means of the pump P2. Thereby the previously generated particles are made to precipitate and are stabilised. In this manner, particles are obtained which may have diameters ranging from a fewμm up to approx. 1 mm, depending upon the process parameters. The oil (or fat etc.) which contains the substance to be encapsulated is included inside the particles. On the outside, the pellets are coated with a Ca alginate layer. They may thus be coated subsequently, if required, as any other Ca alginate particles.

For flushing, the reactor FR may be water-filled via the valves V5 and V7 by means of P2. The water can be drained either by opening the valve KH1 or removed again by pumping out of FR by means of P2 via V6 and V3.

After the pellets have been cured the second process step, i. e. coating, may follow. According to the inventive embodiment, this is done by alternately rinsing the capsules with a cationic and an anionic diluted polymer solution. In between times, washings steps are scheduled. The particles are subjected several minutes to the solutions which may be pumped back into the reservoirs. It is of importance that the capsules are maintained in a kind of fluidised bed, i. e. in a suspended condition, so that the membrane may develop all over. This can be achieved by means of special agitators and/or as illustrated in the present figure by the tangential infeed of the solutions at a relatively high velocity which is to amount to several metres per second at the pipe outlet opening. The liquids may be temperature equalised by means of the corresponding heat exchangers WT2. Upon completion of the coating process, the finished membrane capsules are washed and flushed out of the reaction vessel. Subsequently, a drying step may be carried out through which the capsules are dehydrated. The selected drying method is mainly determined on the basis of the encapsulated material.

In the embodiment shown in FIG. 1 the material is guided from FR into the second reactor BTR upon opening of the valve KH1. Here, the particles are washed first. For this purpose, the pellets are decanted by opening the valves KH2 and VT. BTR has a conical shape in order to facilitate this decanting process. Alternatively, the excess liquid can be pumped off by the pump P4 via the valves V25 and V9. The DI water which is required for washing is pumped into the reactor BTR by means of the pump P3 via the valves V8, V22, and V26. The washing water may then be either decanted or pumped off as previously described. The first coating reagent, the polycation 1, is supplied from the reservoir PK1 into the coating reactor BTR by opening of the valves V11, V22, and V26 and by pumping by means of the pump P2. After a certain level has been reached in BTR, the solution may be circulated around by closing of V22 and V23 and opening of V24 and V26. By agitation with the agitator R4, the particles are kept in suspension during all processes. After the formed gel particles have dwelled for several minutes in the coating bath the solution is pumped back to PK1 by closing of V26 and opening of V23 and V10. The pellets are then washed with DI water by opening of V8, V22, and V26, with the water being pumped off again upon opening of V9, V25 by means of the pump P4. By switching the corresponding valves, the reactor BR is then flushed in a similar cycle with the detergent solution from the reservoir E and then with the first polyanion from the reservoir PA1, followed by 2 to 3 washing cycles. Subsequently, the reactor is supplied with a second polycationic solution from the reservoir PK2, which will then be pumped back into same. This process sequence will then be repeated in the same manner with the corresponding reagents from the reservoirs PS2 (second polyanion) or PA3 (third polyanion) until the desired membrane has been formed. The membrane capsules are then flushed out of the reactor by opening the ball valve KH2 and with the valve VT in the appropriate position.

The pellets obtained in this manner may then be supplied to a drying step. Very good results will be achieved in a fluidized bed air drying process.

The entire plant may be cleaned and disinfected with conventional cleaning agents by corresponding filling-in and pumping off of the solutions.

1. A method and plant for the production of microcapsules for the immobilisation of chemical active substances, proteins, living cells, and/or micro organisms on an industrial scale, characterised in that
the material to be encapsulated is solved, suspended, or emulsified in a liquid which cannot be mixed with water and in this form is delivered from a mixing tank into a reactor where pellets are produced therefrom in a coacervate process, which seal the material and which subsequently may be coated in the same and/or another vessel by repeated flushing with appropriate reagents which are supplied from various reservoirs.

2. The method according to claim 1, characterised in that
it comprises several or all of the following steps which may also be repeated several times:
solving, suspending, or emulsifying the material to be encapsulated in a basic substance which cannot be mixed with water;
delivering this basic substance, i. e. suspension, emulsion, or solution into a reaction vessel;
emulsifying of this suspension, emulsion, or solution at an increased temperature in a further liquid mixture which contains e. g. gelatine, water, and glycerine;
metering of an enveloping solution e. g. of Na alginate to the now solution;
lowering the temperature of the new mixture;
metering a reagent, e. g. calcium chloride, which precipitates the enveloping solution (e. g. alginate);
precipitating the drops;
rinsing and suspending the pellets which have been generated by the precipitation in a washing liquid;
flushing of the pellets with a polycationic polymer solution and forming a cationic charge on the pellet surface;
washing of the pellets in washing liquid;
washing of the pellets in a detergent solution;
flushing of the pellets with an polyanionic polymer solution and forming an anionic charge on the pellet surface;
rinsing and suspending the pellets which have been generated by precipitation in a washing liquid;
separating the pellets from the surrounding liquid medium;
drying of the pellets.

3. The method according to claim 1, characterised in that
the basic substance is a fat or an oil.

4. The method according to claim 1, characterised in that
the material to be encapsulated/the basic substance, i. e. suspension, emulsion, or solution is conveyed by a mechanic means, preferably a worm conveyor or a pump, into a reaction vessel.

5. The method according to claim 1, characterised in that
the material to be encapsulated/the basic substance, i. e. suspension, emulsion, or solution is conveyed pneumatically into a reaction vessel.

6. The method according to claim 1, characterised in that
the drops in the phase which cannot be mixed with water contain the material to be immobilised.

7. The method according to claim 1, characterised in that
the drops are coated with enveloping material by precipitation.

8. The method according to claim 1, characterised in that
this enveloping reagent is an alginate salt.

9. The method according to claim 1, characterised in that
the enveloped drops are maintained in suspension in the reaction solutions.

10. The method according to claim 1, characterised in that
the enveloped drops are maintained in suspension in the reaction solutions by agitating.

11. The method according to claim 1, characterised in that
the enveloped drops are maintained in suspension in the reaction solutions by the flow rate of the surrounding medium.

12. The method according to claim 1, characterised in that
the enveloped drops are coated by flushing with suitable polymer solutions.

13. The method according to claim 1, characterised in that
the enveloped drops are maintained in suspension during coating.

14. The method according to claim 1, characterised in that
the enveloped drops are maintained in suspension during coating by agitating.

15. The method according to claim 1, characterised in that
the enveloped drops are maintained in suspension during coating by the flow rate of the surrounding medium.

16. The method according to claim 1, characterised in that
the coated pellets comprise an envelope which completely seals the core and the encapsulated material.

17. The method according to claim 1, characterised in that
the envelope of the coated pellets consists of one or more radially arranged layers.

18. The method according to claim 1, characterised in that
layers of the envelope may be zones of different density.

19. The method according to claim 1, characterised in that
the coated pellets may be stored and used in the non-dried condition, i. e. in the moist condition.

20. The method according to claim 1, characterised in that
the coated pellets may be freeze-dried.

21. The method according to claim 1, characterised in that
the coated pellets may be air-dried.

22. The method according to claim 1, characterised in that
solutions which are employed for precipitation and/or coating are used either as concentrates or in a ready to use diluted form.

23. The plant according to claim 1 which operates in accordance with a method according to claim 1, characterised in that
it comprises several of the following major components:
mixing/emulsifying tank (EG) for the basic substance which cannot be mixed with water and the material to be immobilised;
mixing tank (WG) for the reactions solutions;
reservoir (FB) for the precipitation reagent;
reservoir (A) for the enveloping reagent;
reservoir (E) for a washing solution, preferably a detergent;
reservoirs (PK1, PK2, PA1, PA2, PA3) for the coating polymers;
reaction-emulsifying vessel (FR) for the production of the particles;
reaction vessel (BTR) for coating and separation of the enveloped particles;
device for drying the coated pellets;
heat exchangers (WT1, WT2) for temperature equalising of the reaction vessels;
pumps (P1, P2, P3, P4) and valves (V1, V2, . . . ) for filling and draining the reaction vessels, as well as ball valves (KH1, KH2, KH);
pneumatic valves and components;
heating/cooling thermostats.

24. The plant according to claim 23, characterised in that
it operates according to FIG. 1 and/or its components are arranged and/or interconnected according to FIG. 1.
